(19) United States
(12) Patent Application Publication
(10) Pub. No.: US 2026/0047771 A1
(43) Pub. Date: Feb. 19, 2026

(54) BIOELECTRICAL IMPEDANCE ANALYZER CAPABLE OF OBTAINING BONE MINERAL DENSITY

(71) Applicant: STARBIA MEDITEK CO., LTD., TAICHUNG CITY (TW)

(72) Inventor: KUEN-CHANG HSIEH, TAICHUNG CITY (TW)

(21) Appl. No.: 19/369,952

(22) Filed: Oct. 27, 2025

Related U.S. Application Data
(63) Continuation-in-part of application No. 17/687,022, filed on Mar. 4, 2022, now abandoned.

(30) Foreign Application Priority Data
Mar. 5, 2021 (TW) 110107862

(51) Int. Cl. A61B 5/0537; A61B 5/00

(52) U.S. Cl. CPC A61B 5/0537; A61B 5/6825; A61B 5/6829; A61B 5/7271; A61B 5/742

(57) ABSTRACT

A bioelectrical impedance analyzer includes a plurality of electrode sets attached to a subject to generate a bioelectrical signal, a signal processing unit electrically connected to the electrode sets and configured to convert the bioelectrical signal into a resistance and a reactance of the subject, and an estimation unit electrically connected to the signal processing unit and configured to obtain the bone mineral density of the subject according to the following formula: BMD=a+b×(R/H)+d×(Xc/H), where BMD represents the bone mineral density; a, b, and d are weighting coefficients; R represents the resistance of the subject; Xc represents the reactance of the subject; H represents a height of the subject. Accordingly, the bioelectrical impedance analyzer can achieve advantages of reducing measurement costs and rapid measurement.

### CROSS-REFERENCE TO RELATED APPLICATIONS

This application is a Continuation-in-Part of copending application Ser. No. 17/687,022, filed on Mar. 4, 2022, which is hereby expressly incorporated by reference into the present application.

### BACKGROUND OF THE INVENTION

### 1. Field of the Invention

The present invention relates to bone mineral density evaluation technology, and more particularly, to a bioelectrical impedance analyzer capable of obtaining bone mineral density.

### 2. Description of the Related Art

Most of the current bone mass inspection methods are measured according to the different absorption levels of ionized radiation by bone and soft tissue. Among them, the use of dual energy X-ray absorptiometry (DXA) to express bone mineral density (BMD) by mineral mass (g/cm²) is widely accepted.

However, although the method of applying DXA to measuring BMD is accepted by everyone, because DXA measurement is expensive, and its measurement location must be implemented in a hospital or related professional institution, it cannot be applied to home health care.

In addition, each measurement of DXA takes tens of minutes. Therefore, the current method of measuring bone mineral density using DXA is costly and time-consuming, and it is not convenient to use.

### SUMMARY OF THE INVENTION

It is a primary objective of the present invention to provide a bioelectrical impedance analyzer, which can obtain bone mineral density of a subject, and is low in cost, fast, and convenient to use.

To attain the above objective, the bioelectrical impedance analyzer of the present invention comprises a plurality of electrode sets, a signal processing unit, and an estimation unit. The electrode sets are attached to hands and feet of a subject to generate a bioelectrical signal. The signal processing unit is electrically connected to the electrode sets and configured to convert the bioelectrical signal into a resistance and a reactance of the subject. The estimation unit is electrically connected to the signal processing unit and configured to obtain bone mineral density of the subject according to a following formula: BMD=a+b×(R/H)+d×(Xc/H), where BMD represents the bone mineral density; a, b, and d are weighting coefficients; R represents the resistance of the subject; Xc represents the reactance of the subject; H represents a height of the subject.

It can be seen from the above that the bioelectrical impedance analyzer of the present invention provides advantages of low cost, fast measurement, and convenient operation as compared with the prior art.

Preferably, the estimation unit is electrically connected to an output unit that is configured to generate a personalized evaluation report based on an estimation result. Through the personalized evaluation report, the subject not only better understands his or her own bone mineral density but also is enabled to perform personal health management. The personalized evaluation report can be displayed on a display screen or printed out by a printer.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is a block diagram of a bioelectrical impedance analyzer of the present invention;

FIGS. 2-4 are schematic drawings, showing that the subject is instructed to perform the measurement in a supine, sitting and standing position; and

FIG. 5 is a GLM regression analysis diagram of the bioelectrical impedance analyzer of the present invention.

### DETAILED DESCRIPTION OF THE INVENTION

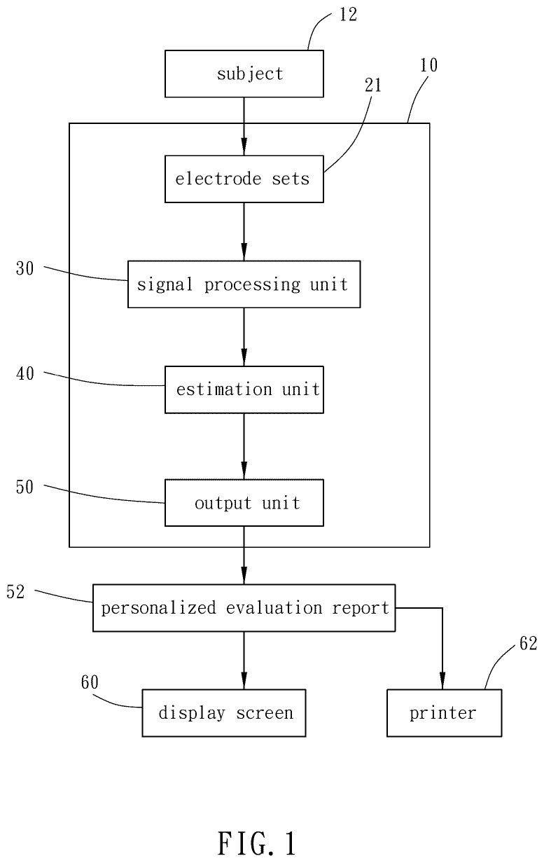

Please refer to FIG. 1, a bioelectrical impedance analyzer 10 of the present invention comprises a plurality of electrode sets 21, a signal processing unit 30, and an estimation unit 40.

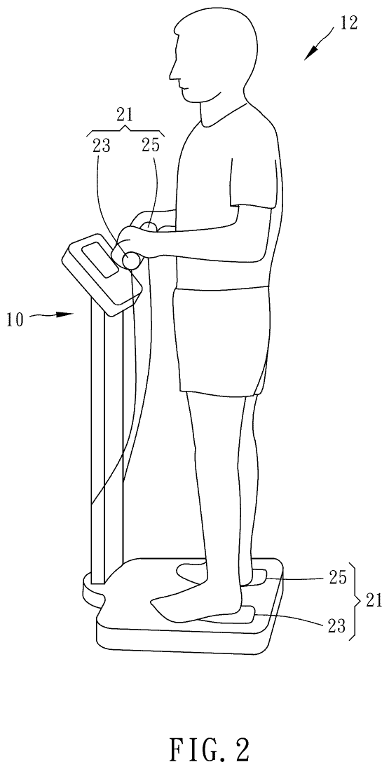

The electrode sets 21 are attached to hands and feet of a subject 12 to generate a bioelectrical signal. In this embodiment, the subject 12 operates the bioelectrical impedance analyzer 10 in a standing posture (as shown in FIG. 2) so as to reduce evaluation errors. As an example, the number of the electrode set 21 is two, and the electrode sets 21 are attached to the right hand and the right foot of the subject 12 to form a measurement loop. Each electrode set 21 includes a sensing electrode 23 and a current electrode 25, and the sensing electrode 23 and the current electrode 25 are spaced apart by 5 cm. Based on the characteristics of bioelectrical impedance measurement, such a configuration can enhance evaluation accuracy.

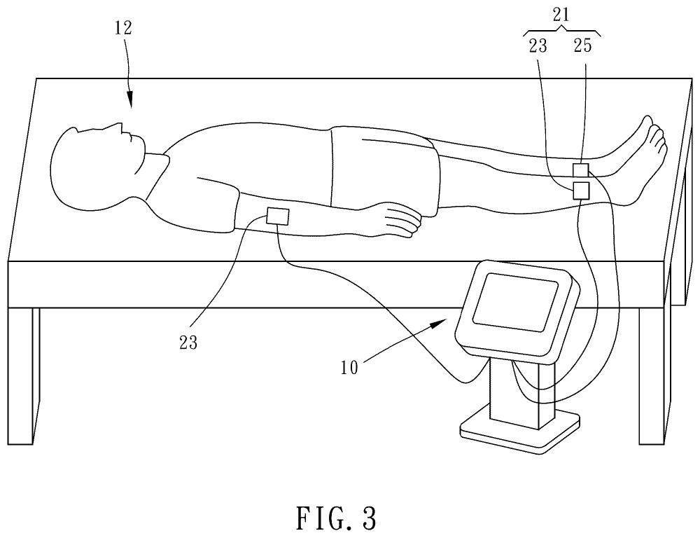

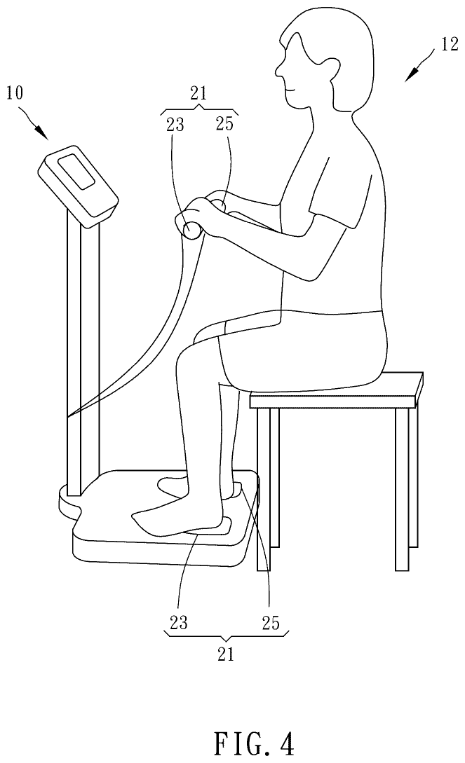

In other embodiments, the subject 12 may also operate the bioelectrical impedance analyzer 10 in a supine posture (as shown in FIG. 3) or a sitting posture (as shown in FIG. 4). The electrode sets 21 may respectively contact the subject's both hands, both feet, right hand and left foot, left hand and right foot, or left hand and left foot. Under different measurement modes, the number of the electrode set 21 may exceed two, and the sensing electrode 23 and the current electrode 25 may be spaced apart by 1-5 cm or 5-10 cm. Accordingly, the number of the electrode set 21 and the operation posture of the subject 12 can be adjusted according to actual requirements, and are not limited to this embodiment described herein.

The signal processing unit 30 is electrically connected to the electrode sets 21 and configured to convert the bioelectrical signal into a resistance and a reactance of the subject 12.

The estimation unit 40 is electrically connected to the signal processing unit 30 and configured to obtain bone mineral density of the subject 12 according to a following formula: BMD=a+b×(R/H)+d×(Xc/H), where BMD represents the bone mineral density; a, b, and d are weighting coefficients; R represents the resistance of the subject 12; Xc represents the reactance of the subject 12; H represents a height of the subject 12.

In this embodiment, the bone mineral density is obtained at three body parts, including the whole body, the lumbar spine, and the right upper limb. In other embodiments, other body parts of the subject 12 may also be selected for measurement as needed, and the number of the body parts measured may be increased or reduced according to actual requirements.

In this embodiment, the weighting coefficients a, b, and d are obtained by using dual-energy X-ray absorptiometry (DXA). Specifically, the bone mineral density values of several hundred subjects are measured at three body parts, including the whole body, the lumbar spine, and the right upper limb, and the average values thereof are used to determine the weighting coefficients a, b, and d. In other embodiments, the weighting coefficients a, b, and d may also be obtained by using other instruments such as a quantitative ultrasound device, a quantitative computed tomography (QCT) device, or a conventional X-ray device. In addition, other body parts of the subject 12 may be selected for measurement as needed, and the number of the body parts measured may be increased or reduced according to actual requirements. Therefore, the measuring instrument and the body parts of the subject 12 are not limited to this embodiment described herein.

In this embodiment, the height of the subject 12 is measured by a stadiometer and manually input into the bioelectrical impedance analyzer 10, wherein the precision of the stadiometer is up to 0.5 cm, but is not limited thereto. In this way, the height most consistent with the actual condition of the subject 12 can be obtained, thereby improving evaluation accuracy. In other embodiments, the height of the subject 12 may be directly provided by the subject 12 and transmitted to the bioelectrical impedance analyzer 10 automatically in a wired or wireless manner. Accordingly, the method of obtaining the height of the subject 12 is not limited to this embodiment described herein.

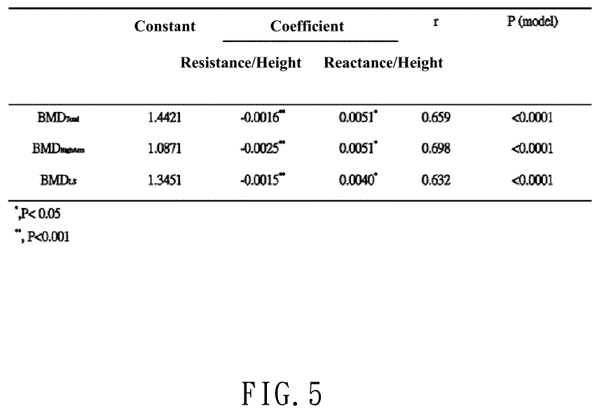

As shown in FIG. 5, in the generalized linear model (GLM) regression analysis, the corresponding weighting coefficients and correlations indicate that the values of (R/H) and (Xc/H) are positively correlated with the measurement results of total bone mineral density (BMDₜₒₜₐₗ), lumbar spine bone mineral density (BMD_{LS}), and right arm bone mineral density (BMD_{right arm}).

It is to be additionally noted that the estimation unit 40 is electrically connected to an output unit 50 configured to generate a personalized evaluation report 52 based on an estimation result. Through the personalized evaluation report 52, the subject 12 not only better understands his or her own bone mineral density but also is enabled to perform personal health management. The personalized evaluation report 52 may be displayed on a display screen 60 or printed out by a printer 62.

In summary, the bioelectrical impedance analyzer 10 of the present invention obtains the resistance, reactance, and height of the subject 12, and the bone mineral density of the subject 12 can then be obtained according to the formula BMD=a+b×(R/H)+d×(Xc/H). Compared with the prior art, the present invention has advantages of low cost, rapid measurement, and convenient use.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A bioelectrical impedance analyzer comprising:
a plurality of electrode sets configured to be attached to hands and feet of a subject to generate a bioelectrical signal;
a signal processing unit electrically connected to the electrode sets and configured to convert the bioelectrical signal into a resistance and a reactance of the subject; and
an estimation unit electrically connected to the signal processing unit and configured to obtain bone mineral density of the subject according to a following formula: BMD=a+b×(R/H)+d×(Xc/H), where BMD represents the bone mineral density; a, b, and d are weighting coefficients; R represents the resistance of the subject; Xc represents the reactance of the subject; H represents a height of the subject.

2. The bioelectrical impedance analyzer as claimed in claim 1, further comprising an output unit electrically connected to the estimation unit and configured to generate a personalized evaluation report based on an estimation result.

3. The bioelectrical impedance analyzer as claimed in claim 2, wherein personalized evaluation report is displayed on a display screen.

4. The bioelectrical impedance analyzer as claimed in claim 1, wherein the bioelectrical impedance analyzer is configured to be operated with the subject in a supine, standing, or sitting posture selectively.
